(19) United States
(12) Patent Application Publication
(10) Pub. No.: US 2024/0311527 A1
(43) Pub. Date: Sep. 19, 2024

(54) SYSTEMS AND METHODS FOR SELF-ASSEMBLY AND DESIGN OF LATTICES FOR OPTICAL METAMATERIALS

(71) Applicants: Arizona Board of Regents on Behalf of Arizona State University, Tempe, AZ (US); UNIVERSITÀ CA' FOSCARI, Venezia (IT); SAPIENZA UNIVERSITY OF ROME, Rome (IT)

(72) Inventors: Petr Sulc, Scottsdale, AZ (US); Michael Matthies, Mesa, AZ (US); Flavio Romano, Venezia (IT); John Russo, Rome (IT); Hao Liu, Tempe, AZ (US)

(21) Appl. No.: 18/606,649

(22) Filed: Mar. 15, 2024

Related U.S. Application Data
(60) Provisional application No. 63/490,711, filed on Mar. 16, 2023.
(60) Provisional application No. 63/509,999, filed on Jun. 23, 2023.

(51) Int. Cl. G06F 30/20; G16B 15/10

(52) U.S. Cl. CPC G06F 30/20; G16B 15/10

(57) ABSTRACT

A computer-implemented system uses multiscale modeling and optimization algorithms to design DNA nanostructures that self-assemble into a target structure, such as a tetrastack lattice. The system converts a target structure into a Boolean Satisfiability problem and models nanostructure units as “patchy” nanostructure models, where patches encode kinetic properties that affect how each nanostructure model connects with other nanostructure models. The system iteratively simulates self-assembly of a plurality of nanostructure models into a nanostructure assembly model, and compares the nanostructure assembly model with the target structure to determine a set of optimal parameters (such as patch type assignments) for the nanostructure units that reliably result in self-assembly into the target structure.

### CROSS REFERENCE TO RELATED APPLICATIONS

This is a non-provisional application that claims benefit to U.S. Provisional Application Ser. No. 63/490,711, filed on Mar. 16, 2023, and U.S. Provisional Application Ser. No. 63/509,999, filed on Jun. 23, 2023, which are herein incorporated by reference in their entireties.

### GOVERNMENT SUPPORT

This invention was made with government support under N00014-20-1-2094 awarded by the Office of Naval Research. The government has certain rights in the invention.

### FIELD

The present disclosure generally relates to design of photonic metamaterials, and in particular, to a system and associated method for self-assembly and design of lattices for optical metamaterials.

### BACKGROUND

Construction of metamaterials is of importance to the fields such as optical computing, photonics and plasmonics. However, development of these metamaterials can be challenging as they need to be precisely oriented and structured in order to be useful in these fields.

Sophisticated statistical mechanics approaches and human intuition have demonstrated the possibility to self-assemble—mostly in silico—complex lattices or finite size constructs. The proposed strategies are quite often subject to unpredicted (and unpredictable) traps, associated with kinetic slowing down (gelation, glass transition) as well as to competing ordered structures. Theoretical predictions also crush against the possibility to encode using the currently available library of nano and micron-sized particles the desired inter-particle interaction potential.

It is with these observations in mind, among others, that various aspects of the present disclosure were conceived and developed.

### BRIEF DESCRIPTION OF THE DRAWINGS

The present patent or application file contains at least one drawing executed in color. Copies of this patent or patent application publication with color drawing(s) will be provided by the Office upon request and payment of the necessary fee.

FIG. 1 shows an overview of a computer-implemented system for designing DNA nanostructures, as applied for implementation of tetrastack lattice;

FIG. 2 is a simplified diagram showing an example computing system for implementation of the system of FIG. 1 discussed herein;

FIG. 3A is a simplified diagram showing a general workflow of the system of FIG. 1, as applied for implementation of tetrastack lattice;

FIG. 3B is a simplified diagram further showing a general workflow of the system of FIG. 3A;

FIGS. 4A-4C are a series of process flow diagrams showing a method for designing DNA nanostructures that can be implemented by the system of FIG. 1 corresponding to the workflows shown in FIGS. 3A and 3B;

FIGS. 5A-5E are a series of graphical representations showing example computational outputs for a sequence design of DNA nanostructures with oxDNA simulation of an assembled lattice; and

FIGS. 6A-6D show experimental characterization of the fabricated tetrastack lattice, where FIGS. 6A and 6B correspond with octahedral “building blocks” and FIGS. 6C and 6D correspond with icosahedron “building blocks”.

Corresponding reference characters indicate corresponding elements among the view of the drawings. The headings used in the figures do not limit the scope of the claims.

### SUMMARY

A system outlined herein includes a processor in communication with a memory, the memory including instructions encoded thereon and executable by the processor to: access target assembly information including target unit cell information, the target unit cell information encoding an architecture of a target unit cell of a target nanostructure assembly; iteratively construct a nanostructure system model that represents each nanostructure model of a plurality of nanostructure models as a patchy volumetric shape having a plurality of patches, the nanostructure system model representing kinetic interactions between respective patches of each nanostructure model and each nanostructure model of the plurality of nanostructure models belonging to a species of nanostructure model; iteratively simulate a self-assembly process of a nanostructure assembly model from a plurality of nanostructure models of the nanostructure system model, the self-assembly process being governed by simulation of kinetic interactions between patches of each nanostructure model of the plurality of nanostructure models; and determine an optimal patch type assignment for each respective species of nanostructure model of the nanostructure system model such that a resultant nanostructure assembly model matches the target nanostructure assembly.

The memory can further include instructions executable by the processor to: translate the target unit cell information into a set of Boolean satisfiability clauses that encode structural and unit design requirements to form the target unit cell from a plurality of nanostructure units of a nanostructure system, each nanostructure unit of the plurality of nanostructure units belonging to a species of nanostructure unit; and populate an interaction matrix based on the set of Boolean satisfiability clauses for the target unit cell, the interaction matrix representing a patch type assignment for each respective species of nanostructure unit, the patch type assignment representing kinetic properties that affect compatibility between two or more patch types. In some examples, the memory can further include instructions executable by the processor to: apply a Boolean satisfiability solver to the set of Boolean satisfiability clauses for the target unit cell; and assign, based on an output of the Boolean satisfiability solver, a patch type to each respective patch of each respective species of nanostructure unit of the nanostructure system that satisfies the set of Boolean satisfiability clauses.

The memory can further include instructions executable by the processor to: generate, for a species of nanostructure unit of a nanostructure system and based on an interaction matrix representing a patch type assignment for the nanostructure unit, the nanostructure model of the nanostructure system model that represents the species of nanostructure unit having patch type assignments based on the interaction matrix. The nanostructure model can include one or more patches corresponding with a physical location along a nanostructure unit belonging to an appropriate species of nanostructure unit of the nanostructure system.

The memory can further include instructions executable by the processor to: compare a unit cell model of the nanostructure assembly model to the target unit cell information; and adjust one or more parameters of the nanostructure system model based on comparison between the unit cell model of the nanostructure assembly model and the target unit cell information.

In some examples, the target nanostructure assembly is a tetrastack lattice structure. The nanostructure model can represent a DNA nanostructure, and the nanostructure model can include one or more patches, each patch of the one or more patches corresponding to an overhang of the DNA nanostructure. The nanostructure system model can include an interaction matrix that represents interactions between a first patch of a first nanostructure model having a first patch type and a second patch of a second nanostructure model having a second patch type. The first patch and the second patch can be complementary with one another and can correspond to complementary single-stranded DNA overhangs of the first nanostructure model and the second nanostructure model.

The memory can further include instructions executable by the processor to: simulate application of a mixing process to the plurality of nanostructure models of the nanostructure system model; and simulate application of an annealment process to the plurality of nanostructure models of the nanostructure system model.

In another aspect, a method outlined herein includes: accessing target assembly information including target unit cell information, the target unit cell information encoding an architecture of a target unit cell of a target nanostructure assembly; iteratively constructing a nanostructure system model that represents each nanostructure model of a plurality of nanostructure models as a patchy volumetric shape having a plurality of patches, the nanostructure system model representing kinetic interactions between respective patches of each nanostructure model and each nanostructure model of the plurality of nanostructure models belonging to a species of nanostructure model; iteratively simulating a self-assembly process of a nanostructure assembly model from a plurality of nanostructure models of the nanostructure system model, the self-assembly process being governed by simulation of kinetic interactions between patches of each nanostructure model of the plurality of nanostructure models; and determining an optimal patch type assignment for each respective species of nanostructure model of the nanostructure system model such that a resultant nanostructure assembly model matches the target nanostructure assembly.

The method can further include: translating the target unit cell information into a set of Boolean satisfiability clauses that encode structural and unit design requirements to form the target unit cell from a plurality of nanostructure units of a nanostructure system, each nanostructure unit of the plurality of nanostructure units belonging to a species of nanostructure unit; and populating an interaction matrix based on the set of Boolean satisfiability clauses for the target unit cell, the interaction matrix representing a patch type assignment for each respective species of nanostructure unit, the patch type assignment representing kinetic properties that affect compatibility between two or more patch type.

The method can further include: applying a Boolean satisfiability solver to the set of Boolean satisfiability clauses for the target unit cell; and assigning, based on an output of the Boolean satisfiability solver, a patch type to each respective patch of each respective species of nanostructure unit of the nanostructure system that satisfies the set of Boolean satisfiability clauses.

The method can further include: generating, for a species of nanostructure unit of a nanostructure system and based on an interaction matrix representing a patch type assignment for the nanostructure unit, the nanostructure model of the nanostructure system model that represents the species of nanostructure unit having patch type assignments based on the interaction matrix, and the nanostructure model including one or more patches corresponding with a physical location along a nanostructure unit belonging to an appropriate species of nanostructure unit of the nanostructure system.

The method can further include: comparing a unit cell model of the nanostructure assembly model to the target unit cell information; and adjusting one or more parameters of the nanostructure system model based on comparison between the unit cell model of the nanostructure assembly model and the target unit cell information.

Further, the method can include: simulating application of a mixing process to the plurality of nanostructure models of the nanostructure system model; and simulating application of an annealment process to the plurality of nanostructure models of the nanostructure system model.

In some examples, the nanostructure model represents a DNA nanostructure and includes one or more patches, each patch of the one or more patches corresponding to an overhang of the DNA nanostructure. The nanostructure system model can include an interaction matrix that represents interactions between a first patch of a first nanostructure model having a first patch type and a second patch of a second nanostructure model having a second patch type, the first patch and the second patch being complementary with one another and corresponding to complementary single-stranded DNA overhangs of the first nanostructure model and the second nanostructure model.

In a further aspect, a non-transitory computer readable medium can include instructions encoded thereon which are executable by a processor to implement aspects of the methods outlined herein, including: accessing target assembly information including target unit cell information, the target unit cell information encoding an architecture of a target unit cell of a target nanostructure assembly; iteratively constructing a nanostructure system model that represents each nanostructure model of a plurality of nanostructure models as a patchy volumetric shape having a plurality of patches, the nanostructure system model representing kinetic interactions between respective patches of each nanostructure model and each nanostructure model of the plurality of nanostructure models belonging to a species of nanostructure model; iteratively simulating a self-assembly process of a nanostructure assembly model from a plurality of nanostructure models of the nanostructure system model, the self-assembly process being governed by simulation of kinetic interactions between patches of each nanostructure model of the plurality of nanostructure models; and determining an optimal patch type assignment for each respective species of nanostructure model of the nanostructure system model such that a resultant nanostructure assembly model matches the target nanostructure assembly.

### DETAILED DESCRIPTION

### 1. Introduction

The experimental realization of nanoscopic and mesoscopic structures with precise geometry is one of the “holy grails” of nanotechnology. Among the different strategies devised, self-assembly (where details of a structure to be obtained or otherwise assembled are encoded in nanostructure units or “building blocks”) is one of the most promising. However, the nanostructure units require a careful design, currently generated through painstaking trial-and-error procedures, to self-assemble into the desired structure with high yield without encountering kinetic traps or unwanted byproducts.

An important applicative avenue for this technology is generation of superlattices with photonic applications: if assembled at the nanoscale, these materials would enable manipulation of visible light for information processing. Several 3D nano-lattice structures that pose band-gaps in the visible light domain have been proposed. Self-assembly provides one of the most promising approaches for constructing such lattices, although the presence of competing metastable states requires fairly complex design of particles to be able to assemble only into the target structure. Recently, different types of 3D lattices have been assembled either from DNA coated colloids or DNA origami.

Expanding the types of nanocrystal lattices that can be self-assembled can enable development of new types of functionalized materials with applications in sensing, imaging, energy conversion, waveguide manufacturing, and optical computing. As such, there is a need in the art for a common designing platform that is general enough to target multiple lattices and, at the same time, yield solutions that avoid kinetic traps and assemble only the target structure.

Here, the present disclosure introduces a new modeling-driven design pipeline that uses optimization methods and multiscale simulations to design self-assembling DNA nanoparticles that can self-assemble into a target structure. The modeling pipeline accounts both for positive and negative design, assuring that DNA nanoparticles can form the target structure, while also ensuring that the DNA nanoparticles avoid forming competing alternative states previously identified in the simulation.

As a test case, the present disclosure focuses on a tetrastack (also known as pyrochlore) lattice as a target structure. This target structure is of particular interest as it has been identified as having an omnidirectional photonic band gap, which is both wide and robust with respect to defects in the lattice. The photonic properties of the tetrastack lattice are very similar to those of the diamond cubic lattice. This similarity is due to the close relationship between the two structures: the tetrastack is the lattice formed by the contacts in the diamond structure (and vice versa).

While prior work has succeeded in hierarchical self-assembly of the cubic diamond lattice at the microscale level from polystyrene colloidal particles, and more recently from tetrahedral DNA origami cages, the tetrastack lattice geometry has so far not been successfully experimentally realized by self-assembly.

The present disclosure demonstrates the versatility of methods outlined herein by designing two different DNA origami wireframe nanostructures that bind to each other through single-stranded overhangs and robustly assemble into the tetrastack superlattice.

### II. Computational Pipeline for Inverse Design

### II-A. Overview

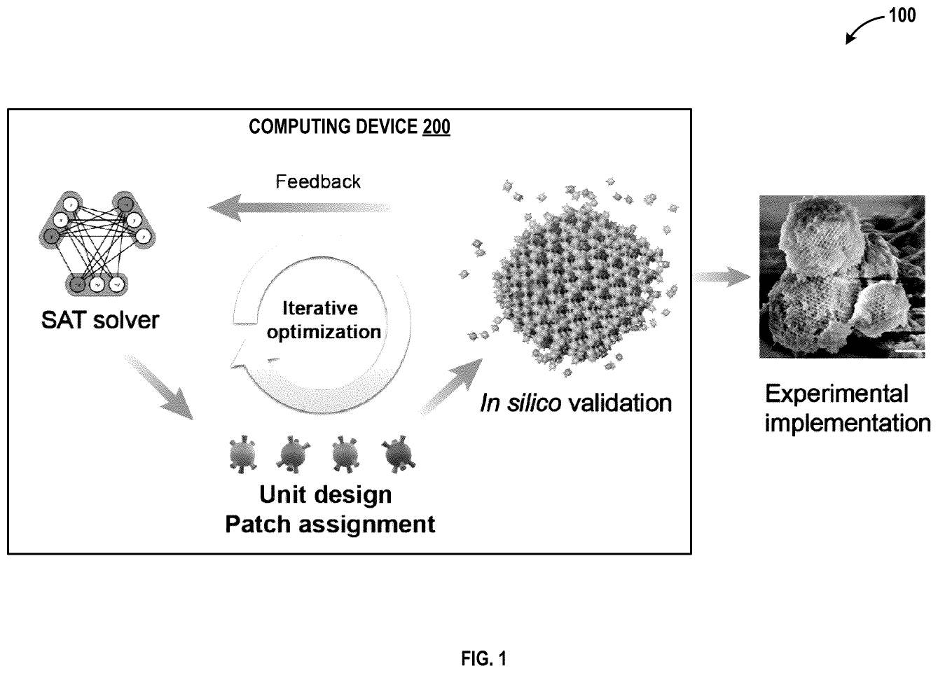

FIG. 1 shows an overview of a computer-implemented system (hereinafter, system 100) for generating DNA nanoparticle designs that self-assemble into a target structure (e.g., a tetrastack lattice). With a Boolean Satisfiability (SAT) solver, the system 100 generates an interaction matrix for the target structure and specified assembling species. The system 100 then customizes nanostructure units (in other words, building blocks) with a preferred topology and defines patch positions specifying a pairing set based on the interaction matrix. In some embodiments, “patch types” can be discussed in terms of colors which can aid understanding when graphical representations are displayed with corresponding colors, however note that patch types can be encoded or otherwise identified or denoted within the system 100 using any suitable identification scheme. Following customization of nanostructure units with patch positions, the system 100 can then simulate self-assembly of the nanostructure units with a patchy particle model for in silico validation of the produced designs on interactions and nanostructure units. Based on the simulation results, the system 100 can generate or otherwise provide feedback for the interaction matrix. The system 100 can then adjust or otherwise re-generate the interaction matrix based on the feedback. The system 100 can then continue with iterative optimization by repeating the process in view of the feedback, including generating new designs for the nanostructure units with a (new) interaction matrix and simulating self-assembly of the nanostructure units until the nanostructure units can reliably generate the target structure. For cases with successful self-assembly in the simulation, the system 100 can transfer the designs into DNA origamis to examine wet-lab assembly of the targeted structure.

### II-A. Computer-Implemented System

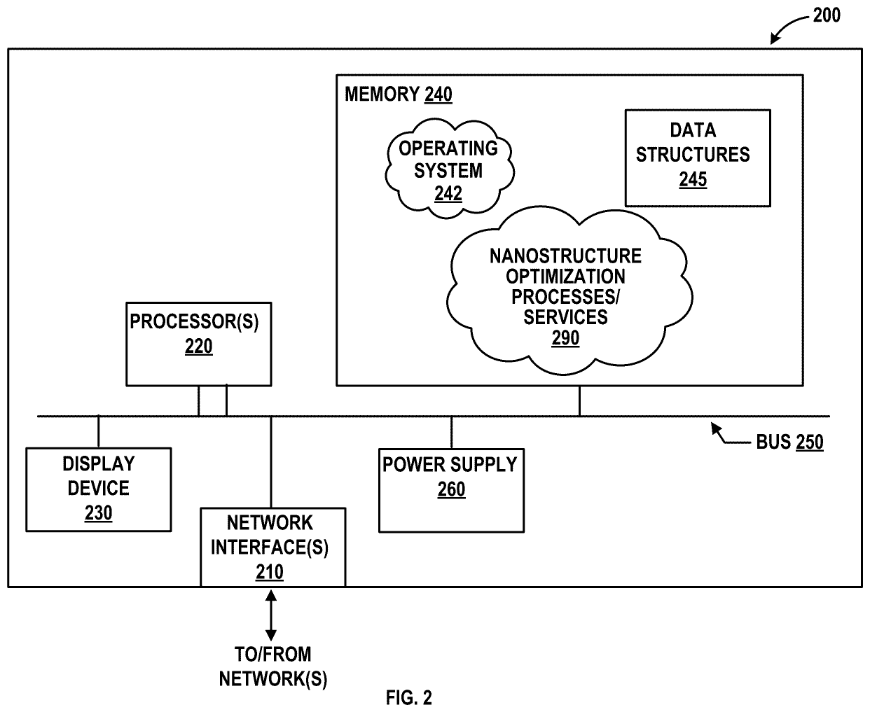

FIG. 2 is a schematic block diagram of an example device 200 that may be used with one or more embodiments described herein, e.g., as a component of the system 100 applying methods outlined above.

Device 200 includes one or more network interfaces 210 (e.g., wired, wireless, PLC, etc.), at least one processor 220, and a memory 240 interconnected by a system bus 250, as well as a power supply 260 (e.g., battery, plug-in, etc.). Device 200 can also include or otherwise communicate with a display device 230 that displays results of the methods, such as renderings of nanostructure units and resulting self-assembled structures.

Network interface(s) 210 include the mechanical, electrical, and signaling circuitry for communicating data over the communication links coupled to a communication network. Network interfaces 210 are configured to transmit and/or receive data using a variety of different communication protocols. As illustrated, the box representing network interfaces 210 is shown for simplicity, and it is appreciated that such interfaces may represent different types of network connections such as wireless and wired (physical) connections. Network interfaces 210 are shown separately from power supply 260, however it is appreciated that the interfaces that support PLC protocols may communicate through power supply 260 and/or may be an integral component coupled to power supply 260.

Memory 240 includes a plurality of storage locations that are addressable by processor 220 and network interfaces 210 for storing software programs and data structures associated with the embodiments described herein. In some embodiments, device 200 may have limited memory or no memory (e.g., no memory for storage other than for programs/processes operating on the device and associated caches). Memory 240 can include instructions executable by the processor 220 that, when executed by the processor 220, cause the processor 220 to implement aspects of the system and the methods outlined herein.

Processor 220 includes hardware elements or logic adapted to execute the software programs (e.g., instructions) and manipulate data structures 245. An operating system 242, portions of which are typically resident in memory 240 and executed by the processor, functionally organizes device 200 by, inter alia, invoking operations in support of software processes and/or services executing on the device. These software processes and/or services may include nanostructure optimization processes/services 290, which can include aspects of the methods and/or implementations of various modules described herein. Note that while nanostructure optimization processes/services 290 is illustrated in centralized memory 240, alternative embodiments provide for the process to be operated within the network interfaces 210, such as a component of a MAC layer, and/or as part of a distributed computing network environment.

It will be apparent to those skilled in the art that other processor and memory types, including various computer-readable media, may be used to store and execute program instructions pertaining to the techniques described herein. Also, while the description illustrates various processes, it is expressly contemplated that various processes may be embodied as modules or engines configured to operate in accordance with the techniques herein (e.g., according to the functionality of a similar process). In this context, the term module and engine may be interchangeable. In general, the term module or engine refers to model or an organization of interrelated software components/functions. Further, while the nanostructure optimization processes/services 290 is shown as a standalone process, those skilled in the art will appreciate that this process may be executed as a routine or module within other processes.

### II-B. Nanostructure Optimization Framework

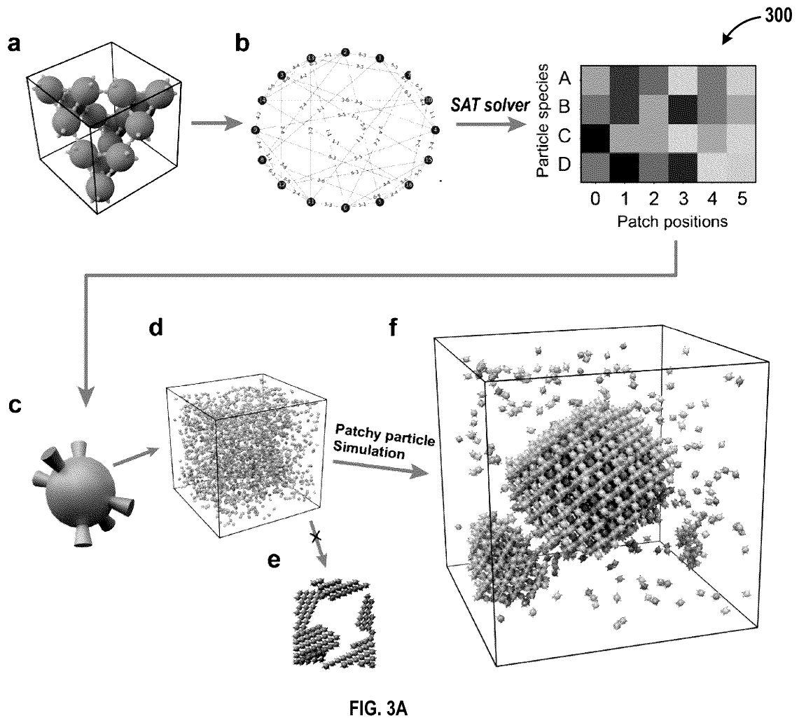

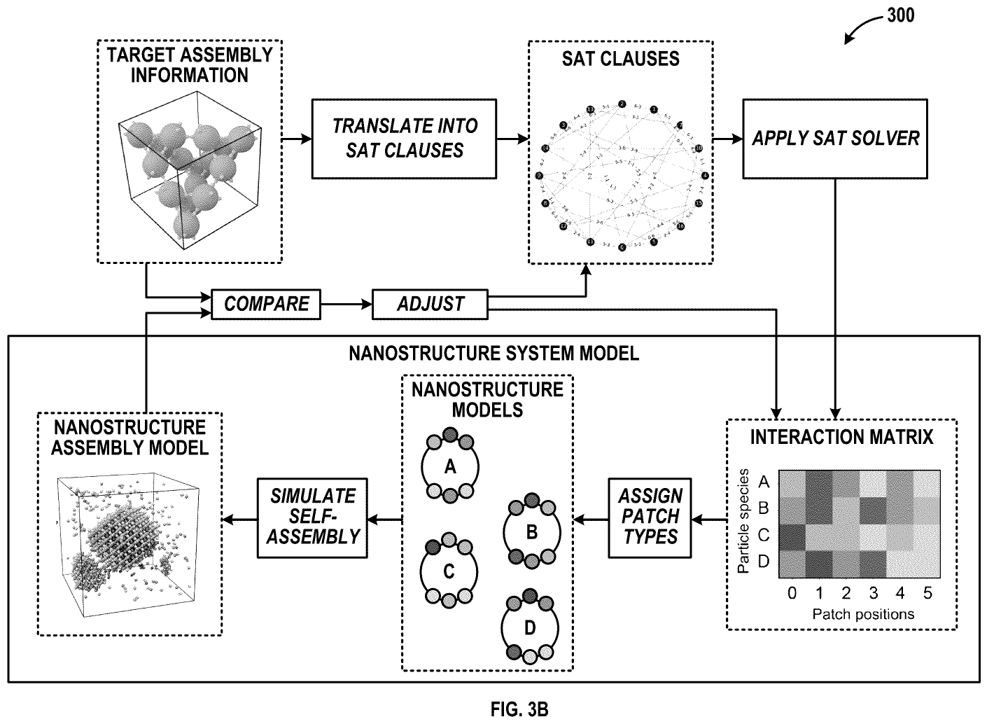

FIGS. 3A and 3B are simplified diagrams showing a general overview of a framework 300 applied by the system 100 for designing DNA nanostructures, as applied for self-assembly of a target structure, e.g., a tetrastack lattice. The system 100 accesses target assembly information that encodes an architecture of a target unit cell of the target structure (in this case, a tetrastack lattice) (point a of FIG. 3A), and translates the target assembly information into a set of Boolean satisfiability (SAT) clauses (point b of FIG. 3A) to fulfill the structural and unit design requirements for the target unit cell. Using the SAT clauses, the system 100 can construct a nanostructure system model for designing and simulating self-assembly of nanostructure models, which represent nanostructure units with the aim of self-assembly into the target unit cell.

To construct the nanostructure system model based on the target assembly information, the system 100 applies an SAT solver which generates an interaction matrix. The interaction matrix corresponds to assignment of patchy interactions (e.g., interactions between “patches” of the nanostructure unit to be designed, where each “patch” of a nanostructure unit can interact with another “patch” of another nanostructure unit). The interaction matrix enables the system 100 to further generate nanostructure models of the nanostructure system model that represent each species of nanostructure unit in the form of a monomeric particle design (point c of FIG. 3A). Each nanostructure model generally represents the corresponding species of nanostructure unit in the form of a patchy volumetric shape (referred to herein as a “patchy particle model”) that includes a plurality of patches, each patch having a patch type that is assigned based on the interaction matrix. The system 100 can then simulate self-assembly of the nanostructure models into a nanostructure assembly model of the nanostructure system model by modeling kinetic interactions between patches of the nanostructure models of all designed assembling species (point d of FIG. 3A) with a dedicated model in stoichiometric ratio within a simulation box. Simulating interaction of the nanostructure models informs about self-assembly result of the nanostructure units after screening for a number of temperatures. With inferior patch assignment (point e of FIG. 3A), unpreferred states could form and should be avoided. In contrast, successful self-assembly (point f of FIG. 3A) could also emerge at correct temperatures. Following generation of the nanostructure assembly model, the system 100 can compare the nanostructure assembly model with the target unit cell and adjust parameters of the nanostructure system model (e.g., the SAT clauses and/or interaction matrix, among other properties such as quantity of nanostructure models or attraction force) before repeating the self-assembly simulation.

The system 100 can determine optimal patch type assignments for nanostructure models of the nanostructure system model by iteratively repeating the above process until the resultant nanostructure assembly model reliably matches the target unit cell while avoiding undesirable configurations. The optimal patch type assignments can then be used to construct real-world nanostructure units which can be tested under an actual self-assembly process, and may be used in real-world applications.

### II-C. Interaction Matrix and Boolean Satisfiability Solver

As such, the present disclosure outlines a multiscale approach to design and verify in silico DNA nanoparticles that can reliably self-assemble into a target structure such as a tetrastack lattice. The target unit cell of the tetrastack lattice structure (FIGS. 3A and 3B) includes 16 individual particles, where each particle has six neighbors. The neighbor position on the particle surface is close to the positions of vertices on opposite faces of an icosahedron. Therefore, DNA origami (icosahedral) wireframe structures can be used as the basic building unit, where complementary single-stranded overhangs connect the neighboring particles in the lattice.

For simulation and design of the DNA origami wireframe nanostructures, a nucleotide-level coarse-grained model, oxDNA, which has been shown to reproduce the structural, mechanical and thermodynamic properties of single and double-stranded DNA. However, this model is still too slow to simulate the kinetics of assembly of individual monomers into the lattice. Hence, to study assembly kinetics, a coarser model was developed where the individual DNA nanostructures are represented as patchy particle “spheres” (point c of FIG. 3A). In this example, each type of patch is assigned a color. If two patches represented by two colors are compatible, they are considered to correspond to complementary single-stranded DNA overhangs and, in the patchy representation, to a short range attractive potential. Note that while the examples outlined herein denote patch types in terms of color, the system 100 may use other suitable identification schemes to represent patch types and compatibility between patch types. The patchy particle spheres interact with excluded volume interaction to prevent two particles from overlapping with each other. In some implementations, the oxDNA model can be used to parameterize the patchy particle model.

The goal of the inverse design procedure employed by the system 100 is to assign an interaction matrix representing the nanostructure units (s), where the interaction matrix sets the attraction strength between any pair of DNA overhangs (e.g., represented with patch types in the coarser patchy model) so that the nanostructure unit(s) self-assembly into the target structure without kinetic traps or alternative free-energy minima that would lead to misassembled structures or defects in the lattice. To test the system 100, multiple patchy particle simulations were conducted across a range of temperatures to probe assembly kinetics for each possible solution, specified by the colorings assigned to respective patches on the respective particles representing the nanostructure unit(s), and the list of compatible colors. Previous research has identified that a solution that uses only one species of particle will lead in simulation to a misassembled state. At high temperatures, the assembly can remain in gas phase, whereas at low temperatures the assembly can form a quenched glassy state. At intermediate temperatures (where one would hope to observe nucleation and assembly of the ordered lattice), the assembly was found to form misassembled states (shown in FIG. 2) which are stabilized by two bonds formed between two complementary pairs of patches on two patchy particles.

The number of possible ways to design interaction matrices between patchy particles explodes combinatorically with an increasing number of possible colors and particle species, which in turn makes the search of the design space very challenging. As discussed, to generate an interaction matrix that can avoid these trapped states, the SAT-assembly design framework embodied by the system 100 maps the inverse design problem to a Boolean Satisfiability problem (SAT). SAT is a well-studied NP-complete problem for which highly efficient solvers are available, enabling solutions to the design problem. These solutions can be represented as a set of binary variables that specify which patch type (represented by color) is assigned to which particle and which colors can interact. SAT further specifies restrictions that the interactions need to satisfy in terms of binary logic clauses, which can be composed of AND, OR and NOT operations on the set of binary variables. These restrictions can enforce, for example, that each color can only have one complementary color, each patch can only be assigned one color, and that the patchy particles can be arranged into a unit lattice so that all the patches on each particle are bound to a patch of complementary color. Thus, as a positive design task, the system 100 can specify in terms of binary variables and logic clauses that the target structure is an energy minimum of the patchy particle system, and let the SAT solver find color interactions and a patch color assignment scheme that satisfy this condition. The SAT can also enforce negative design requirements: no two particles can bind to each other by more than one bond at a time, which explicitly prevents the nanostructure system model from forming the misassembled state that was previously identified in the molecular dynamics simulation.

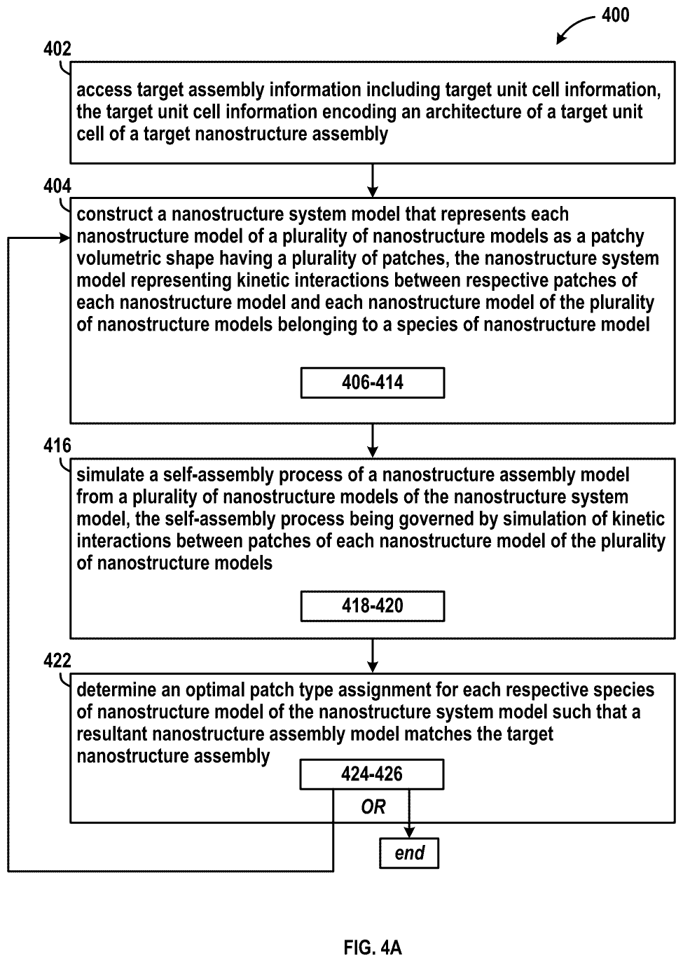

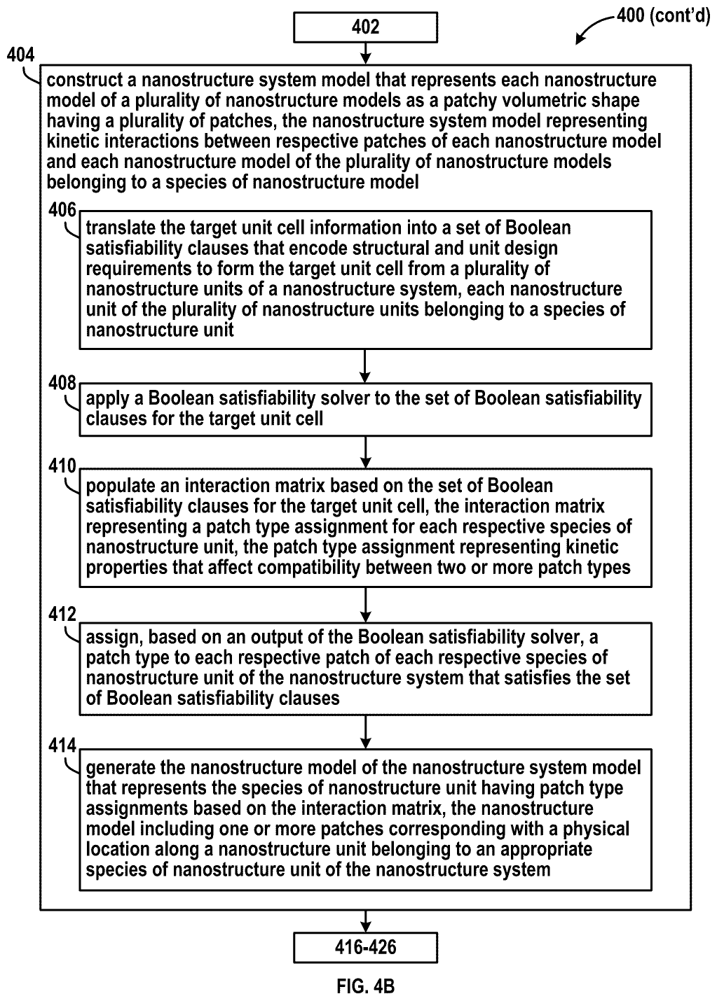

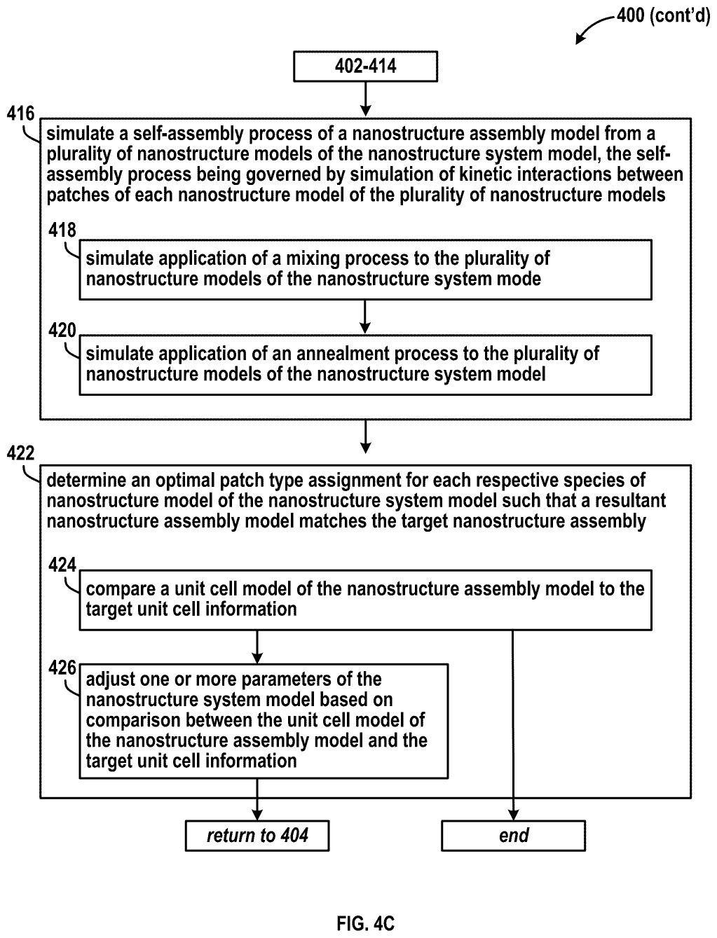

For a target structure such as the tetrastack lattice design, the SAT solver proved that no solution exists that satisfies the positive and negative design conditions if only one patchy particle species is used. There are possible solutions with two distinct particle species, but such solutions always require that each particle is able to bind to another particle of the same species. Since the goal is to realize the patchy particle as a DNA origami interacting via single-stranded overhangs, the system 100 can further impose a requirement that a particle cannot form any bond with a particle of the same species in order to prevent possible aggregations or blocking by unpaired staple strands when each DNA nanostructure type is prepared individually by mixing the DNA origami scaffold and staple strands. With this additional requirement, the SAT solver identifies that the smallest number of distinct particle species required is four. Since simulation of assembly kinetics shows that a larger number of distinct colors generally leads to faster assembly, a solution with 24 different colors is employed (12 pairs of complementary colors, with the interaction matrix between patches shown in FIGS. 4A and 3B). This solution is verified to homogeneously nucleate into a tetrastack crystal in molecular dynamics simulations at a range of tested temperatures.

### II-D. Computer-Implemented Method

A method 400 outlined herein and shown in FIGS. 4A-4C for optimizing patch type assignment and other parameters of a nanostructure system model may be implemented using computing device 200 (e.g., as part of nanostructure optimization processes/services 290) in accordance with the system 100 shown in FIG. 1 and the framework 300 shown in FIGS. 3A and 3B. The method 400 corresponds with FIGS. 3A and 3B and their corresponding discussion in the present disclosure.

Referring to FIG. 4A, step 402 of method 400 includes accessing target assembly information including target unit cell information, the target unit cell information encoding an architecture of a target unit cell of a target nanostructure assembly.

Step 404 of method 400 can include (iteratively) constructing a nanostructure system model that represents each nanostructure model of a plurality of nanostructure models as a patchy volumetric shape having a plurality of patches, the nanostructure system model representing kinetic interactions between respective patches of each nanostructure model and each nanostructure model of the plurality of nanostructure models belonging to a species of nanostructure model. Step 404 can include sub-steps 406-414 shown in FIG. 4B.

As shown in FIG. 4B, steps 406-414 are sub-steps of step 404 of method 400 outlined above. Step 406 includes translating the target unit cell information into a set of Boolean satisfiability clauses that encode structural and unit design requirements to form the target unit cell from a plurality of nanostructure units of a nanostructure system, each nanostructure unit of the plurality of nanostructure units belonging to a species of nanostructure unit. Step 408 includes applying a Boolean satisfiability solver to the set of Boolean satisfiability clauses for the target unit cell. Step 410 includes populating an interaction matrix based on the set of Boolean satisfiability clauses for the target unit cell, the interaction matrix representing a patch type assignment for each respective species of nanostructure unit, the patch type assignment representing kinetic properties that affect compatibility between two or more patch type. Step 412 includes assigning, based on an output of the Boolean satisfiability solver, a patch type to each respective patch of each respective species of nanostructure unit of the nanostructure system that satisfies the set of Boolean satisfiability clauses.

Referring back to FIG. 4A, step 416 of method 400 includes (iteratively) simulating a self-assembly process of a nanostructure assembly model from a plurality of nanostructure models of the nanostructure system model, the self-assembly process being governed by simulation of kinetic interactions between patches of each nanostructure model of the plurality of nanostructure models. Steps 418-420 shown in FIG. 4C can be sub-steps of step 416. Step 418 includes simulating application of a mixing process to the plurality of nanostructure models of the nanostructure system model. Step 420 includes simulating application of an annealment process to the plurality of nanostructure models of the nanostructure system model.

Referring back to FIG. 4A, step 422 of method 400 includes determining an optimal patch type assignment for each respective species of nanostructure model of the nanostructure system model such that a resultant nanostructure assembly model matches the target nanostructure assembly. Step 422 can include sub-step 424 and, sub-step 426 when applicable, both shown in FIG. 4C. Step 424 includes comparing a unit cell model of the nanostructure assembly model to the target unit cell information. If the nanostructure assembly model reliably matches the target assembly expressed by the target unit cell information (and reliably avoids other sub-optimal configurations), then the process can end and the optimal parameters and patch type assignment for a nanostructure unit would correspond with the nanostructure system model. However, if the nanostructure assembly model does not reliably match the target assembly expressed by the target unit cell information (and/or does not reliably avoid other sub-optimal configurations), then step 426 may be applied. Step 426 can include adjusting one or more parameters of the nanostructure system model based on comparison between the unit cell model of the nanostructure assembly model and the target unit cell information. Steps 404-426 can be iteratively repeated until the nanostructure assembly model reliably assembles into the target assembly expressed by the target unit cell information (and reliably avoids other sub-optimal configurations).

In some examples, the nanostructure model represents a DNA nanostructure. In such an example, the nanostructure model includes one or more patches, where each patch of the one or more patches corresponds to an overhang of the DNA nanostructure. The nanostructure system model can include an interaction matrix that represents interactions between a first patch of a first nanostructure model having a first patch type and a second patch of a second nanostructure model having a second patch type, the first patch and the second patch being complementary with one another and corresponding to complementary single-stranded DNA overhangs of the first nanostructure model and the second nanostructure model.

### II-E. Implementation Examples and Experimental Validation

In one example implementation, the coarse-grained oxDNA model and design tool oxView were employed to design DNA nanostructures to represent the patchy particles with wireframe DNA origami, where patches correspond to single-stranded overhangs with spacers. Two DNA wireframe origami designs were considered: an icosahedral origami shape and an octahedral origami shape. In the icosahedral origami shape, each “patch” corresponds to three single-stranded overhangs (“handles”) placed in the vertex of the DNA wireframe origami, making the overall geometry fully compatible with the corresponding patchy particle model. Each overhang has a 15-nucleotide poly-T spacer followed by the 8-nucleotide long binding region at the 3′ flanking end. The binding region sequences are the same on each of the three sequences in the patch, so that there is no imposed orientational control over binding of the two patches. The assigned binding regions were optimized such that for each complementary pair, the binding free-energy of complementary sequences was as close as possible for all twelve binding pairs, while making the binding between overhangs that are not supposed to interact as unfavorable as possible.

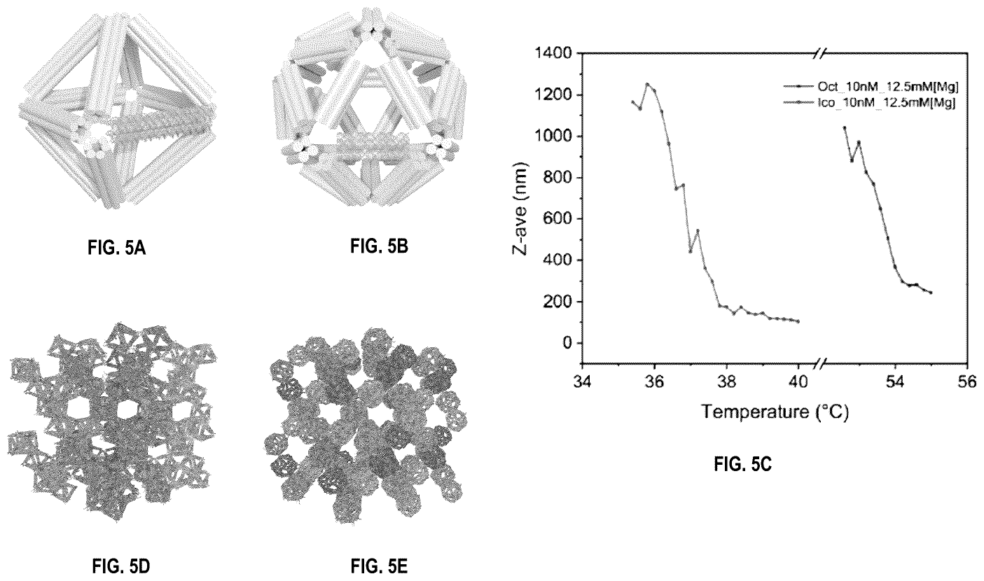

FIGS. 5A-5E show transferring the computational output to the sequence design of DNA nanostructures with oxDNA simulation of the assembled lattice. FIGS. 5A and 5B show octahedral and icosahedral DNA origamis selected for the experimental implementation. Both structures are equipped with multi-helical bundle edges to ensure the structural rigidity. FIG. 5C shows dynamic light scattering spectrums measure the size change of the assemblies in solution for the systems programmed to form tetrastack lattice with octahedral and icosahedral DNA origami respectively. FIGS. 5D and 5E show screenshots of the assembled and relaxed tetrastack lattice in oxDNA and visualized with oxView.

To demonstrate the robustness of the overhang-driven assembly and inverse-design strategy, an octahedral origami design was also employed where the vertex positions did not perfectly correspond to the patch positions. This in turn required that each handle is sufficiently long to adapt to the imposed geometry, which is not compatible with octahedra touching their vertices. Hence, the handles were designed with longer poly-T spacers (22 nucleotides) at each vertex to ensure that the DNA origamis could arrange into the tetrastack lattice, and used a 9-nucleotide long binding region. Patchy particle simulations with patches placed in the octahedron vertex positions verified that the design with four particles species and 24 colors is still capable of assembling into a tetrastack lattice structure.

Both icosahedral and octahedral designs were tested in a large-scale simulation with the oxDNA model. To investigate the mechanical stability and design the position and lengths of handle sequences accordingly, a tetrastack lattice cluster was assembled measuring 2×2×2 unit cells in each dimension (total: 128 DNA origami, corresponding to over two million nucleotides in the simulation). Molecular dynamics simulations were conducted for both designs (icosahedron and octahedron units) at 293 K, and were used to calculate the mean structure along with its root mean square fluctuations. The movement trajectory of the center of mass (COM) was assessed for each DNA origami incorporated into the lattice and was superimposed onto the mean structure. A qualitative comparison of the relative positions of the COM trajectory and the mean structure indicates that the lattice assembled using the proposed origami design satisfies the tetrastack geometry and is mechanically stable. The extra single-stranded scaffold loop in the octahedral origami design was also verified to not interfere with the targeted tetrastack geometry.

### III. Lattice Assembly and Characterizations

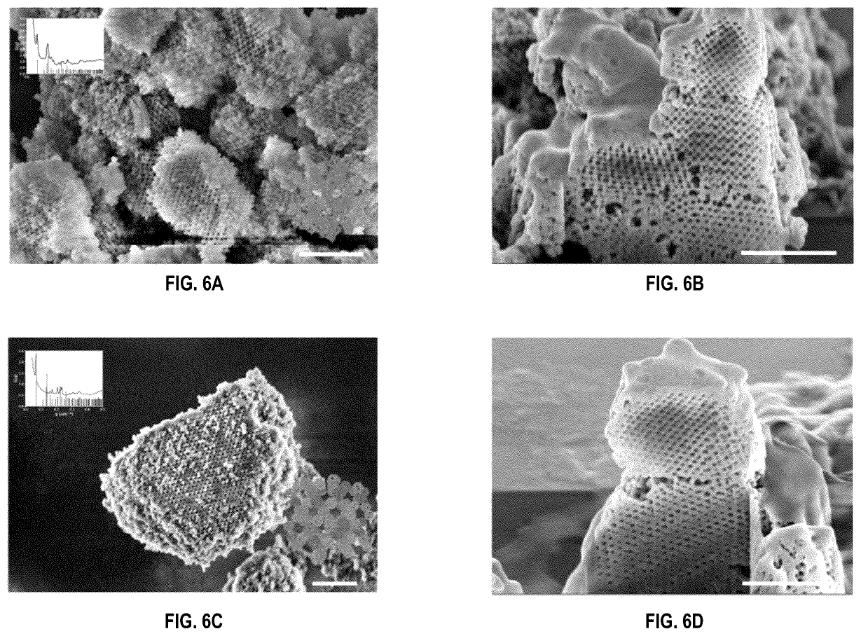

FIGS. 6A-6D show experimental characterization of the fabricated tetrastack lattice in terms of a representative SEM image of the assembled tetrastack lattice with octahedral (FIGS. 6A and 6B) and icosahedron (FIGS. 6C and 6D) building blocks and the associated cross-section of the lattice created by focused ion beam.

For each of the designed DNA origami, each species was prepared in a separate PCR tube by thermal annealing, after which the excess free staple strands were removed. The four different origami species were then mixed together and annealed over a temperature ramp.

The size change of the nanostructure system model was monitored with a fast-annealing protocol using Dynamic Light Scattering (DLS). The measured spectrum enables approximate identification of the temperature range at which monomers start associating (T1) and the temperature where the size of the assemblies reaches a stable size (T2). A customized annealing protocol with a slow ramping rate from T1 to T2 was then employed, after incubation of the nanostructure system model at a slightly higher temperature to dissociate any bonds between monomers. The annealing process, during which the mixed origami nanoparticles nucleate and further crystallize, requires at least one week for the superlattice to emerge, and both elongating and fine-tuning the annealing protocol should give rise to superlattices with enlarged sizes and improved qualities.

Assembly of octahedral and icosahedral origami nanostructure systems happen at different temperature ranges, because of differences in particle geometry, patch distribution, and binding strength (FIG. 6B). Experiments show that a higher binding strength is required for the octahedral system to assemble into the target structure, presumably because the icosahedron is a more preferred geometry for optimal patch positioning, as supported by coarse-grained models.

For characterization, the annealed sample was coated with a thin layer of silica to preserve the structural details for electron microscopy. SEM was used to visualize the deposited silica-DNA hybrid structure. Representative results are shown in FIGS. 6A-6D. The assembly conditions that affect the lattice formation (besides annealing time) were optimized based on the feedback of SEM characterization, including origami concentration and ionic strength (in this case, magnesium ion concentration). Limited by the high binding strength, the octahedral system annealed best with the concentration of origami and magnesium being 10 nM and 25 mM respectively, to ensure the structural integrity of the nanostructure itself during lattice assembly. For the icosahedral origami it was determined (using a slow temperature ramp around the melting point) that concentrations of 10 nM origami and 25 mM magnesium produced the best superlattice, with increased ionic strength allowing larger lattices to emerge. For both nanostructure systems, clear periodicity corresponding to the tetrastack lattices was observed with size around 1 μm for the octahedral system and even larger for the icosahedral system. On average, the octahedral DNA origami produces smaller lattice grains and more poly-crystalline aggregates. The internal structures of the selected lattice grains were further investigated with Focused Ion Beam (FIB) cross-sectional analysis. It appears that internally associated long-range order persists without obvious assembly defects observed, confirming the successful experimental realization of the tetrastack lattice (FIGS. 6A-6D). The stacking of multiple silicated lattices as the cross-section indicates could emerge during the silification and later surface deposition for SEM characterization. SAXS measurements were further performed for the assembled lattices: for the octahedral design, a DNA-coated gold nanoparticle was attached inside the origami. Further, the diffraction measurement of the icosahedral lattice was performed. The comparison of measured structure factor shows agreement with the one expected for the tetrastack lattice.

### IV. Summary

The present application outlines a computer-implemented system and associated methods that use multiscale modeling and optimization algorithms to design DNA nanostructures that self-assemble into a target structure, such as a tetrastack lattice. The system can be generalized to also design and guide experimental realization of other target structures including other type of lattices or finite-size multicomponent assemblies. The system and associated methods could also be used to design initial seeding substructures for improving yields and resulting sizes of the seeded nucleation and growth of the target structure, as well as design other sought-after lattice geometries such as clathrates. In particular, the first successful realization of the tetrastack lattice geometry enabled by the systems and methods outlined herein opens a pathway towards optical metamaterials, which can be achieved by coating silica-coated DNA origami lattice structures with additional metal layers with refractive index in the range required for the photonic crystal property.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A system, comprising:
a processor in communication with a memory, the memory including instructions executable by the processor to:
access target assembly information including target unit cell information, the target unit cell information encoding an architecture of a target unit cell of a target nanostructure assembly;
iteratively construct a nanostructure system model that represents each nanostructure model of a plurality of nanostructure models as a patchy volumetric shape having a plurality of patches, the nanostructure system model representing kinetic interactions between respective patches of each nanostructure model and each nanostructure model of the plurality of nanostructure models belonging to a species of nanostructure model;
iteratively simulate a self-assembly process of a nanostructure assembly model from a plurality of nanostructure models of the nanostructure system model, the self-assembly process being governed by simulation of kinetic interactions between patches of each nanostructure model of the plurality of nanostructure models; and
determine an optimal patch type assignment for each respective species of nanostructure model of the nanostructure system model such that a resultant nanostructure assembly model matches the target nanostructure assembly.

2. The system of claim 1, the memory further including instructions executable by the processor to:
translate the target unit cell information into a set of Boolean satisfiability clauses that encode structural and unit design requirements to form the target unit cell from a plurality of nanostructure units of a nanostructure system, each nanostructure unit of the plurality of nanostructure units belonging to a species of nanostructure unit; and
populate an interaction matrix based on the set of Boolean satisfiability clauses for the target unit cell, the interaction matrix representing a patch type assignment for each respective species of nanostructure unit, the patch type assignment representing kinetic properties that affect compatibility between two or more patch types.

3. The system of claim 2, the memory further including instructions executable by the processor to:
apply a Boolean satisfiability solver to the set of Boolean satisfiability clauses for the target unit cell; and
assign, based on an output of the Boolean satisfiability solver, a patch type to each respective patch of each respective species of nanostructure unit of the nanostructure system that satisfies the set of Boolean satisfiability clauses.

4. The system of claim 1, the memory further including instructions executable by the processor to:
generate, for a species of nanostructure unit of a nanostructure system and based on an interaction matrix representing a patch type assignment for the nanostructure unit, the nanostructure model of the nanostructure system model that represents the species of nanostructure unit having patch type assignments based on the interaction matrix.

5. The system of claim 4, the nanostructure model including one or more patches corresponding with a physical location along a nanostructure unit belonging to an appropriate species of nanostructure unit of the nanostructure system.

6. The system of claim 1, the memory further including instructions executable by the processor to:
compare a unit cell model of the nanostructure assembly model to the target unit cell information; and
adjust one or more parameters of the nanostructure system model based on comparison between the unit cell model of the nanostructure assembly model and the target unit cell information.

7. The system of claim 1, the target nanostructure assembly being a tetrastack lattice structure.

8. The system of claim 1, the nanostructure model representing a DNA nanostructure.

9. The system of claim 8, the nanostructure model including one or more patches, each patch of the one or more patches corresponding to an overhang of the DNA nanostructure.

10. The system of claim 1, the nanostructure system model including an interaction matrix that represents interactions between a first patch of a first nanostructure model having a first patch type and a second patch of a second nanostructure model having a second patch type.

11. The system of claim 10, where the first patch and the second patch are complementary with one another and where the first patch and the second patch correspond to complementary single-stranded DNA overhangs of the first nanostructure model and the second nanostructure model.

12. The system of claim 1, the memory further including instructions executable by the processor to:
simulate application of a mixing process to the plurality of nanostructure models of the nanostructure system model; and
simulate application of an annealment process to the plurality of nanostructure models of the nanostructure system model.

13. A method, comprising:
accessing target assembly information including target unit cell information, the target unit cell information encoding an architecture of a target unit cell of a target nanostructure assembly;
iteratively constructing a nanostructure system model that represents each nanostructure model of a plurality of nanostructure models as a patchy volumetric shape having a plurality of patches, the nanostructure system model representing kinetic interactions between respective patches of each nanostructure model and each nanostructure model of the plurality of nanostructure models belonging to a species of nanostructure model;
iteratively simulating a self-assembly process of a nanostructure assembly model from a plurality of nanostructure models of the nanostructure system model, the self-assembly process being governed by simulation of kinetic interactions between patches of each nanostructure model of the plurality of nanostructure models; and
determining an optimal patch type assignment for each respective species of nanostructure model of the nanostructure system model such that a resultant nanostructure assembly model matches the target nanostructure assembly.

14. The method of claim 13, further comprising:
translating the target unit cell information into a set of Boolean satisfiability clauses that encode structural and unit design requirements to form the target unit cell from a plurality of nanostructure units of a nanostructure system, each nanostructure unit of the plurality of nanostructure units belonging to a species of nanostructure unit; and
populating an interaction matrix based on the set of Boolean satisfiability clauses for the target unit cell, the interaction matrix representing a patch type assignment for each respective species of nanostructure unit, the patch type assignment representing kinetic properties that affect compatibility between two or more patch type.

15. The method of claim 14, further comprising:
applying a Boolean satisfiability solver to the set of Boolean satisfiability clauses for the target unit cell; and
assigning, based on an output of the Boolean satisfiability solver, a patch type to each respective patch of each respective species of nanostructure unit of the nanostructure system that satisfies the set of Boolean satisfiability clauses.

16. The method of claim 13, further comprising:
generating, for a species of nanostructure unit of a nanostructure system and based on an interaction matrix representing a patch type assignment for the nanostructure unit, the nanostructure model of the nanostructure system model that represents the species of nanostructure unit having patch type assignments based on the interaction matrix, and the nanostructure model including one or more patches corresponding with a physical location along a nanostructure unit belonging to an appropriate species of nanostructure unit of the nanostructure system.

17. The method of claim 13, further comprising:
comparing a unit cell model of the nanostructure assembly model to the target unit cell information; and
adjusting one or more parameters of the nanostructure system model based on comparison between the unit cell model of the nanostructure assembly model and the target unit cell information.

18. The method of claim 13, the nanostructure model representing a DNA nanostructure, and the nanostructure model including one or more patches, each patch of the one or more patches corresponding to an overhang of the DNA nanostructure.

19. The method of claim 13, the nanostructure system model including an interaction matrix that represents interactions between a first patch of a first nanostructure model having a first patch type and a second patch of a second nanostructure model having a second patch type, the first patch and the second patch being complementary with one another and corresponding to complementary single-stranded DNA overhangs of the first nanostructure model and the second nanostructure model.

20. The method of claim 13, further comprising:
simulating application of a mixing process to the plurality of nanostructure models of the nanostructure system model; and
simulating application of an annealment process to the plurality of nanostructure models of the nanostructure system model.
